(19) United States
(12) Plant Patent Application Publication
(10) Pub. No.: US 2003/0192097 P1
(43) Pub. Date: Oct. 9, 2003

(54) Chrysanthemum plant named 'Chaclair'

(76) Inventor: Jean-Pierre Challet, Nuaille (FR)

Correspondence Address: BURNS, DOANE, SWECKER & MATHIS, L.L.P., P.O. Box 1404, Alexandria, VA, 22313-1404, US

(21) Appl. No.: 10/118,428

(22) Filed: Apr. 9, 2002

(51) Int. Cl. A01H 5/00

(52) U.S. Cl. PLT/288

(57) ABSTRACT

A new and distinct Chrysanthemum cultivar is provided that is the result of a controlled breeding program. Attractive large ball-shaped uniform pure white blossoms are formed in profusion. Such blossoms commonly are approximately 12 cm in diameter when fully open. The growth habit is very compact and nicely branched following pinching. The foliage is dark green and contrasts well with the pure white blossom coloration, and the stems are very strong. The response time is approximately eight weeks. The plant grows well in pots.

### BOTANICAL/COMMERCIAL CLASSIFICATION

Dendranthema grandifloria/Decorative Pot Mum

### VARIETAL DENOMINATION

cv. ‘Chaclair’

### SUMMARY OF THE INVENTION

The present invention comprises a new and distinct cultivar of Chrysanthemum, botanically known as Dendranthema grandiflora, and hereafter is referred to by the cultivar name ‘Chaclair’.

The new cultivar of the present invention was created at Nuaillé, France during the course of a controlled breeding program. The seeds resulting from the cross were sown and small plants were obtained which were physically and biologically different from each other. Selective study resulted in the identification of a single plant of the new cultivar of the present invention.

It was found that the new Chrysanthemum cultivar of the present invention displays:

(a) a very compact growth habit with good branching following pinching,

(b) forms attractive large long-lasting decorative ball-shaped pure white blossoms,

(c) forms very strong stems,

(d) forms attractive dark green leaves, and

(e) an ability to grow well in pots.

The new cultivar can be grown singly or in clumps in pots. It also can be grown in the landscape. The pure white blossoms of good uniformity contrast nicely with the dark green foliage. The blossoms have been found to be long lasting and commonly last approximately three to four weeks on the plant. The plant responds well to pinching and yields a well-branched growth habit.

Asexual reproduction of the new cultivar by the use of cuttings as performed at Nuaillé, France, in a controlled environment has demonstrated that the characteristics of the new cultivar are firmly fixed and are retained through successive generations of asexual propagation.

‘Chaclair’ has not been observed under all possible environmental conditions to date. Accordingly, it is possible that the phenotype may vary somewhat with variations in the environment, such as temperature, light, day length, contact with pesticides and/or subjection to grown regulation treatments.

### BRIEF DESCRIPTION OF THE PHOTOGRAPHS

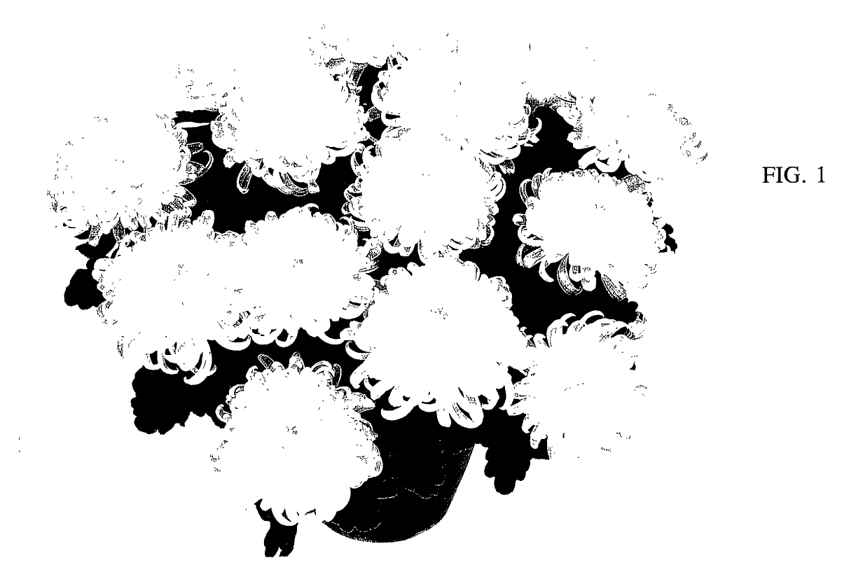

The accompanying FIG. 1 depicts a typical plant of the new ‘Chaclair’ cultivar while growing in a pot in a greenhouse at Nuaillé, France. The attractive pure white ball-shaped blossom configuration and dark green foliage are illustrated. The plant had undergone pinching.

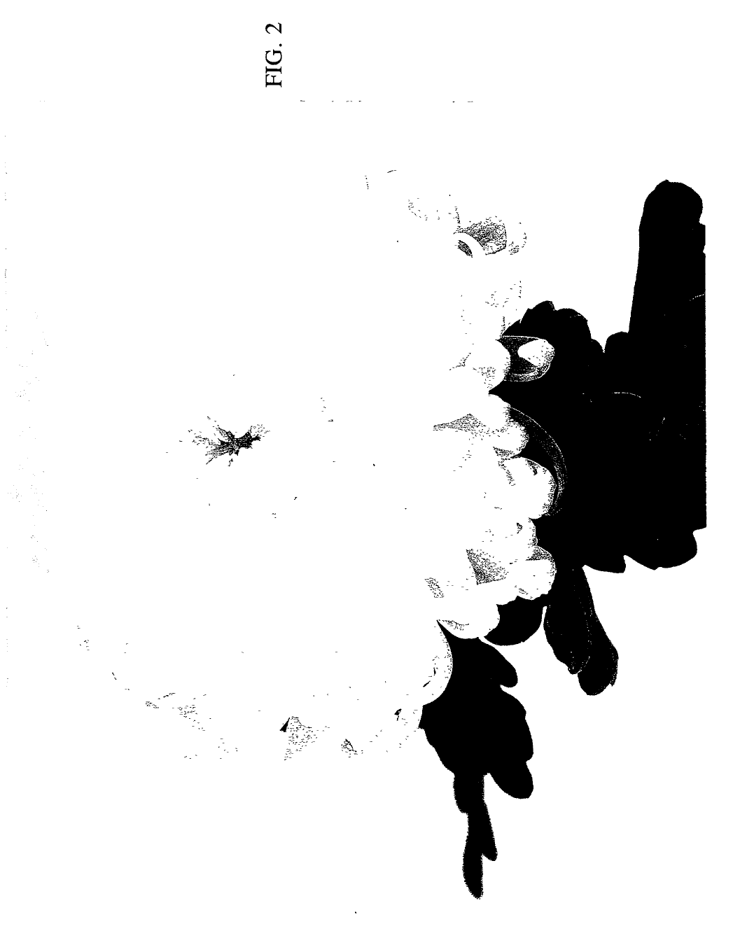

The accompanying FIG. 2 depicts a closer view of a substantially ball-shaped blossom and foliage of the ‘Chaclair’ cultivar.

### DETAILED DESCRIPTION

The plants described were grown in pots in a greenhouse at Nuaillé, France. No growth regulation was used. The growing conditions approximate those commonly utilized for the commercial production of decorative pot mums. The plants had undergone pinching.

General Appearance:

The plant branches nicely following pinching to produce a very compact growth habit.

Foliage:

Attractive dark green (as illustrated).

Stems:

Very strong.

Flowers:

Possess an attractive pure white and very uniform coloration. The configuration is initially somewhat flattened and changes to a generally ball shape with progressing maturity. The diameter is approximately 12 cm when fully expanded.

Flower Duration:

Very long lasting with the blossoms commonly lasting approximately three to four weeks on the plant.

Response Time:

The time between the beginning of the short day period and the flowering date at normal temperature and light intensity is approximately eight weeks.

Usage:

Decorative pot mum with large pure white blossoms.

I claim:

1. A new and distinct cultivar of Chrysanthemum plant that displays:
(a) a very compact growth habit with good branching following pinching,
(b) forms attractive large long-lasting decorative ball-shaped pure white blossoms,
(c) forms very strong stems,
(d) forms attractive dark green leaves, and
(e) an ability to grow well in pots;
substantially as illustrated and described.
